(19) United States
(12) Patent Application Publication
(10) Pub. No.: US 2007/0154565 A1
(43) Pub. Date: Jul. 5, 2007

(54) Method for control of insects on plants and plant tissue

(76) Inventor: Ousama M. Zaghmout, Lorton, VA (US)

Correspondence Address: BIO INTELLECTUAL PROPERTY SERVICES (BIO IPS) LLC, 8509 KERNON CT., LORTON, VA, 22079, US

(21) Appl. No.: 11/707,229

(22) Filed: Feb. 16, 2007

Related U.S. Application Data
(63) Continuation-in-part of application No. 11/179,674, filed on Jul. 13, 2005.
(60) Provisional application No. 60/587,080, filed on Jul. 13, 2004.

(51) Int. Cl. A01N 39/00; A61K 33/40

(52) U.S. Cl. 424/616

(57) ABSTRACT

This invention is directed to a method for control of insects, nematodes, fungi as well as other pathogens on plant tissue which includes applying a solution that includes hydrogen peroxide generating substance alone or in combination with chemicals such as hydrogen peroxide to the plant tissue. Some forms of the invention may utilize an oxalate solution that further includes an acid selected from the group consisting of acetic acid and phosphoric acid. Various structures are used for applying the oxalate solution such as hydraulic spraying, misting, fogging, or injecting the hydrogen peroxide into a commercial cooling system. In one aspect, the oxalate solution is applied to the plant tissue at a variety of stages in the plant production cycle.

### PRIORITY DATES

This application is a continuation-in-part application of application Ser. No. 11/179,674 and filed Jul. 15, 2005 which claims the benefit of Patent Provisional application Ser. No. 60/587,080, filed Jul. 13, 2004 which claims the benefit of Patent Provisional application Ser. No. 60/561,897, filed Apr. 14, 2004 and Patent Provisional Application Ser. No. 60/564,207, filed Apr. 22, 2004 by the present inventor of this application. The entire content of these patent provisional applications are incorporated herein by a reference.

### FEDERALLY SPONSORED RESEARCH

Not Applicable

### SEQUENCE LISTING

Not Applicable

### FIELD OF THE INVENTION

This invention relates to a method of controlling insects in turfgrass, ornamental plants or food crops using an insecticidally effective amount of a substance wherein the substance is a substrate for a hydrogen peroxide generating enzyme such as oxalic acid oxidase, glucose oxidase or citrate oxidase (substrates such as oxalic acid, glucose, citric acid and a like . . . ). This invention also relates to compositions which comprise, in variable quantities, a substrate for hydrogen peroxide generating enzyme alone and/or in combination with a pesticidally active compound in free form or in the form of an agrochemically acceptable salt thereof.

The search for compounds which have a combination of excellent insecticidal activity and low undesirable toxicity is a continuing one because of factors such as the desire for compounds exhibiting greater activity, better selectivity, low undesirable environmental impact, low production cost and effectiveness against insects resistant to many known insecticides.

The compounds and compositions of the present invention are particularly suitable for controlling plant-destructive insects, fungi and nematodes as well as other pathogens in crops of cultivated plants, ornamentals and forestry.

### BACKGROUND OF THE INVENTION

The invention relates to the horticultural and agricultural field and more particularly to compositions and methods for killing, controlling or otherwise impacting the life cycle of insect pests found on agricultural and horticultural products.

Pesticides are an important component to agricultural production throughout the world. Pest control pertains to a wide range of environmental interventions that have their objective to kill or reduce to acceptable level insect pests, plant pathogens and weed populations. Specific control techniques include chemical, physical and biological control mechanisms. It has been estimated that pest annually destroy about 35% of all food crops before they are harvested and another 10-20% loss is incurred after the food is harvested.

Chemical controls include chemical agent pesticides that include herbicides, for the control of weeds, insecticides for the control of insect pests and fungicides for the control of soil and plant pathogens that include bacteria, fungi and viruses. Herbicides account for over half of the pesticides that are uses world wide, with 30-35% of pesticide production in the form of insecticides and the balance for the production of fungicides.

It is important to control populations of insects that affect ornamental and agricultural crops and inflict major damage to the crops resulting in crop loss. Insects can directly affect crop loss by either feeding on the crop itself, thus damaging the plant's ability for producing a fruit or tuber, such as a potato plant or indirectly by either sucking the juices out of some part of a plant that directly affects the aesthetics of the plant, which in the case of ornamental crops such as cut flowers and house plants make the crop unsaleable.

Insects are also a major cause of the spread of infectious disease from plant to plant. As insects feed on the flowers and leaves of the plant, they pick up and transmit potentially deadly pathogenic disease organisms such as bacteria and fungi that are then transmitted to another plant when the insect either crawls on the plant, deposits feces or eats portions of the plant.

The search for compounds which have a combination of excellent insecticidal activity towards target insects and low toxicity towards non-target species is a continuing one because of factors such as the desire for compounds exhibiting greater activity, better selectivity, lower undesirable environmental impact, lack of phytotoxicity to the locus of application, lower production and market cost and higher effectiveness against insects resistant to many known insecticides. In particular, there exists a need for effective control of Coleopteran larvae (grubs) in turfgrass, ornamental plants and food crops. Commercial insecticides, for example chlorpyrifos, carbaryl, acephate, isofenphos, isazophos, diazinon, ethoprop and bendiocarb, have serious deficiencies such as requiring a high application rate to be effective, possessing undesirable toxicity to the mammalian/arian and/or having poor soil mobility. They have also contributed to an unacceptable environmental cycle that directly affects human health and welfare as well as causing direct and indirect environmental damage. Modem day insecticides primarily work by placing a poison or toxin residue on the surface of plant tissue which is likely to be contacted by or eaten by the target insect or by directly spraying the insect pest with the poison compound.

With a typical insecticide, the insect comes into contact with the toxic substance by either being directly sprayed or landing on the residue that has been placed on the plants surface or in the soil in which the plant is ground. The toxin is then either ingested, or enters the insects body through its pores. The toxin then either interferes with the insect's nervous system or other bodily functions such as making it sterile or incapable of eating. Eventually, the insect dies, if the toxin is not deadly enough to produce an immediate kill.

There are several problems that arise from using chemical insecticides. They include resistance, human toxicity, and environment damage.

Since traditional insecticides work on the principal of chemical toxicity, the insect is capable through genetic mutation of developing a resistance to the toxins that affect it. In the insect world, where generations are produced in the span of weeks, the problem of genetic resistance is common. Workers in this field find that within a very short amount of time, many insects that were formally susceptible to certain chemical compounds become resistant or able to tolerate the insectide. Sometimes, within the span of a few years, the chemical either does not produce a kill or the dosage must be increased to produce a kill.

This is why insecticide applicators must cycle their applications of different chemical compounds so as not to allow insects they are trying to control to become accustom to any one chemical compound and ultimately to become immune or resistant to the chemical. This practice of chemical rotation is both times consuming and expensive, since the applicators must, usually, have a minimum of three different chemical compounds for various types of insect pests.

Most chemical insecticides must be used and applied with extreme caution. Typically, the applicator must at all times wear special protective personal protection clothing. This includes the use of respirator and eye protection, as well as chemical impervious coveralls and gloves. Since most insecticides produce a toxic residue and are by nature long lasting and complex compounds, over a period of time, direct exposure to insecticides can lead to human health concerns and in some cases direct exposure to certain insecticides can lead to toxic shock and death.

Due to the very nature of the insecticide that is designed to leave behind a toxic residue on either plant surfaces or in the soil, environmental damage is a direct concern. There has been increasing concern about the impact of groundwater by complex pesticide compounds that do not break down into innocuous substances. It has been documented that pesticide compounds have directly impacted groundwater aquifers and directly threatened environmental security.

In addition to concerns about groundwater impacts, insecticides often impact non-target organisms within the environment that come into contact with the insecticide. These organisms include fish, birds, other non-pest insects, and all forms of animal life. There have been hundreds of documented cases of insecticides such as DDT and others entering the food chain and impacting birds such as the American bald eagle, storks, rainbow trout and others.

Because of the problems associated with the use of traditional chemical insecticides, a need exists for a safe method to control insect pests commonly found in commercial horticulture and agriculture.

An object of the present invention is to provide a safe method of controlling insect populations through either direct killing or by killing of insect larvae and or the elimination of food sources

It is another object of the present invention to provide a method to control insect populations that is safe and convenient to use.

It is another object of the present invention to provide a method of controlling insect pests, which reduces worker exposure to hazardous and toxic compounds.

It is yet another object of the present invention to provide a method of protecting plants from insect infestations through the entire life cycle of the plant.

Although the economic value of the turfgrass industry is difficult to estimate, primarily because much turfgrass acreage is not grown for sale, turfgrass culture in its entirety as an industry contributes significantly to the economy. In the United States, for example, the production, service and maintenance of turfgrass amounts to billions of dollars annually. Protection of existing turfgrass plantings from various pests, including insects, is thus an important concern.

Coleopteran pests are widespread in their habitat. The northern masked chafer, Cyclocephala borealis Arrow, and the southern masked chafer, C. immaculata (Olivier), are native to the United States and are distributed over a wide area east of the Rocky Mountains. May or June beetles, both Phyllophaga spp. Harris, and the oriental beetle, Anomala orientalis Waterhouse, occur throughout Canada and the United States, particularly the eastern half of the United States. The European chafer, Rhizotrogus (Amphimallon) majalis (Razoumowsky), is most problematic in the northeastern United States and in Canada. The cupreous chafer, Anomala cuprea, is a particular problem for crops and turfgrass in Japan.

In the grub stage, the Japanese beetle, Popillia japonica Newman, is undoubtedly the single most important turfgrass-infesting member of the order Coleoptera in the United States. The grub is a major turfgrass pest of golf courses, recreational and industrial parks, school grounds and home lawns. Additionally, it is a major pest as an adult when it feeds on about 300 species of plants, including fruits, vegetables, ornamentals, field and forage crops, and weeds. The beetle's appetite for many ornamental plants greatly increases its pest status in landscape settings. It has a wide geographic distribution in the Northeast and the Midwest of the United States and in Ontario and Quebec in Canada where climatic conditions and large areas of permanent turf favor its development. Popillia japonica is is also a pest in Japan where it attacks highland crops and golf course turfgrass.

It is, therefore, an object of the present invention to provide an effective method for controlling insects in turfgrass, ornamental plants or food crops using an insecticidally effective amount of an oxalate which have unexpectedly high activity against such pests. Because of this unexpectedly high activity, relatively low application rates of these compounds may be employed while control of the pests is maintained. These relatively low application rates, together with the relatively low mammalian toxicity levels possessed by the compounds of the present invention, result in reduced impact on the environment and reduced risk to the applicator, as well as a lower cost of application. Furthermore, by controlling the larvae or grub in the turfgrass or soil environment, a reduction occurs in the subsequent number of adult insects that feed on foliage, flowers, fruits and vegetables above ground.

### SUMMARY OF THE INVENTION

In accordance with the present invention, there are provided insecticidal compositions and methods of using such compositions wherein the compositions comprise an agronomically acceptable carrier and an insecticidally effective amount of, or from about 0.0001% to about 99% by weight of the composition comprising a substance wherein the substance is a substrate for hydrogen-peroxide generating enzyme.

In accordance with the present invention, there are provided fungicidal compositions and methods of using such compositions wherein the compositions comprise an agronomically acceptable carrier and fungicidally effective amount of, or from about 0.0001% to about 99% by weight of the composition comprising a substance wherein the substance is a substrate for hydrogen-peroxide generating enzyme.

In accordance with the present invention, there are provided nematicial compositions and methods of using such compositions wherein the compositions comprise an agronomically acceptable carrier and an nematicidally effective amount of, or from about 0.0001% to about 99% by weight of the composition comprising a substance wherein the substance is a substrate for hydrogen-peroxide generating enzyme.

In one embodiment of the invention, the substrate is an oxalate (Synonyms: Ethanedioic acid, dihydrate; oxalic acid dehydrate), glucose, citric acid or the like. In one aspect, the substance according to this invention may be used in a solution which further comprises hydrogen peroxide to be applied on plant tissue, wherein hydrogen peroxide is in the form of a solution having a concentration of between 0.0001 to 3.00%. In one aspect, the hydrogen peroxide solution has a concentration of between 0.0001 and 1.5%. Some forms of the invention may utilize a hydrogen peroxide solution that further includes an acid selected from the group consisting of acetic acid and phosphoric acid.

In one embodiment, the solution of the substance according to this invention further comprises a chemical inducer of systemic acquired resistance (SAR) such as benzothiadiazole (BTH), 2,6-dichloroisonicotinic acid (INA), Salicylic acid (SA), jasmonic acid, Probenazole, nitric oxide or EDTA, as well as SAR inducers which might potentially induce SAR in a plant (Glynn C. Percival. INDUCTION OF SYSTEMIC ACQUIRED DISEASE RESISTANCE IN PLANTS: POTENTIAL IMPLICATIONS FOR DISEASE MANAGEMENT IN URBAN FORESTRY. Journal of Arboriculture 27(4): July 2001).

In one embodiment, the solution of the substance according to this invention further comprises 5-aminolevulinic acid or a salt thereof.

In one aspect, the solution of the substrate according to this invention further comprises acetosyringone or a like substance.

In one embodiment, the solution of the substrate according to this invention further comprises thidiazuron or a like substance.

In one embodiment, the solution of the substrate according to this invention further comprises a cytokinin or a like substance.

In one embodiment, the solution according to the invention is applied to the plant tissue at a variety of stages in the plant production cycle. Other applications of the invention include applying a mist of the solution over terminal stem cuttings until they reach root sufficiency.

According to this invention, the substrate (e.g., oxalate) may be applied in a liquid or a solid form.

### DESCRIPTION OF THE PREFERRED EMBODIMENT

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

The information provided below is not admitted to be prior art to the present invention, but is provided solely to assist the understanding of the reader. The details of one or more embodiments of the invention are set forth in the accompanying drawings and the description below. Other features, objects, and advantages of the invention will be apparent from the description and drawings, and from the claims.

The method in accordance with the invention utilizes substances for the purpose of killing the most common plant pathogens in greenhouses, lawns, gardens, orchards, forests, and other agricultural crops. In one embodiment, the substance is a substrate for hydrogen-peroxide generating enzyme. In one embodiment, the substrate of the hydrogen-peroxide generating enzyme is an oxalate (oxalic acid), glucose, citric acid or the like. In one embodiment, the substrate for the hydrogen peroxide generating enzyme is applied alone or in combination with other substances as described in this application or that are conventionally used in this field of science.

In this invention, it was discovered that when a dilute solution of oxalate is applied to living plants by spray, mist, fog or immersion (including the root system), the plant pathogens present on the surfaces were either totally eradicated or substantially reduced. The plants suffered no adverse affects from the treatment. Daily treatments with oxalate had no adverse effect upon the health of the plants. There were no signs of discoloration of the flowers on ornamental plants. The most obvious effect was the elimination of the fungi and insects as well as nematodes. The effect of the oxalate upon pathogenic bacteria and viruses are expected to be similar to the effect observed on fungi and insects. The important point to remember is that oxalate is a very powerful pathogen-controlling substance and yet does not harm delicate plant tissues when used at a concentration lethal to the plant pathogens. Other added advantages of oxalate are, the totally innocuous breakdown products of water and hydrogen peroxide which are beneficial for the plant.

Still another surprising advantage of the application of even the moderate concentration of oxalate that will not harm delicate plant tissue is that this moderate concentration will substantially reduce the population of insects in the plant environment. Since insects are a source of plant diseases and even the presence of insects on plants make the plant unsuitable for sale. Plants having an infestation of insects are even worse than being unsuitable for sale in that if any such plants were to reach any retail or wholesale customer for such plants it is unlikely that the customer will quickly purchase any additional plants from the same supplier.

The invention may usually be attained in a method in which an oxalate solution is applied periodically to the plant material throughout the plant production cycle. The method permits the oxalate solution to be applied to the plant material continuously and or periodically during this time period without adversely affecting the plant growth.

More particularly, a solution of oxalate diluted with water to a total oxalate concentration of about 0.0001% to about 99% is preferred. A solution of about 0.1-10% oxalate is preferred for initial application to plant tissue already infected with microorganism while a solution of about 0.1% is preferred for repeated applications intended to protect plant tissue from microbial infection. The addition of hydrogen peroxide or other compounds that are encompassed by this invention to the solution is also part of this invention.

The compositions according to the instant invention generally contain from about 0.0001 to about 100% of oxalate by weight, preferably from 0.0001% to 50%, more preferably from 0.0001% to 25%, most preferably from 0.0001% to 10%. In one aspect of the invention, the compositions according to the instant invention may contain from about 0.0001 to about 95% of oxalate by weight. The remainder of the composition up to 100% comprises a carrier as well as various optional additives such as those hereafter indicated. The addition of hydrogen peroxide or other agriculturally acceptable compounds, encompassed by this invention, to the solution is also part of this invention.

In general the rate of application at the locus is from 0.001 to 10 kilograms of oxalate per hectare (kg/ha), preferably from about 0.01 to about 2 kg/ha, more preferably from about 0.1 to about 1 kg/ha, most preferably from about 0.2 to about 0.8 kg/ha. The compositions of the invention are readily applied by methods know in the agricultural area.

The solution may be delivered to the plant tissue by standard pesticide application techniques. High volumes may be applied by hydraulic spraying and low volumes may be applied by misting or fogging. The solution may also be applied by injecting the oxalate and/or any other substances that are part of this invention into a commercial cooling system. Alternatively, the oxalate and/or any other substances that are part of this invention may be injected into a recirculating subirrigation nutrient system solution to control microbial growth in the irrigation water.

The oxalate solution may be applied to the plant tissue at a variety of stages in the plant production cycle. By injecting oxalate solution alone or in combination with any other substances that are part of this invention into the water that is misted over the terminal stem cuttings until they reach root sufficiency surface microbial contaminants, will be reduced. [This will dramatically reduce the cost of propagating this crop by reducing plant mortality, plant handling, insecticide applications, nematicide applications, and fungicide applications.

Another unexpected result of the greenhouse trials with the oxalate product was the lethal effect upon the insect pest that were present in the greenhouse. The insect pests that were adversely affected by the oxalate included aphids, spider mites, and white fly.

In one embodiment, the composition of the substrate for the hydrogen peroxide generating enzyme(s) further comprises hydrogen peroxide and/or any other systemic acquired resistance inducing compounds such as, but are not limited to, salicylic acid, jasmonic acid, Benzothiadiazole, 2,6-dichloroisonicotinec acid, Probenazole, or nitric oxide.

In one embodiment, the compositions and the methods of the present invention might be practiced with a substrate for hydrogen peroxide generating enzymes and a growth promoting substance such as 5-aminolevulinic acid (U.S. Pat. No. 5,298,482) or Thidiazuron (TDZ). Examples of the salts of 5-aminolevulinic acid include acid addition salts such as the hydrochloride, phosphate, nitrate, sulfate, acetate, propionate, butyrate, valerate, citrate, fumarate, maleate and malate salts as well as metal salts such as the sodium salt, potassium salt and calcium salt.

The composition and the method of use according to this invention may, in one aspect of the invention, further comprise other plant growth regulators, sugars, amino acids, organic acids, alcohols, vitamins, minerals and others. Examples of the plant growth regulators usable here include brassinolides such as epibrassinolides, cholines such as choline chloride and choline nitrate, indolebutyric acid preparations, indoleacetic acid preparations, ethychlozate preparations, 1-naphthylamide preparations, isoprothiolane preparations, nicotinic acid amide preparations, hydroxyisoxasole preparations, calcium peroxide preparations, benzylaminopurine preparations, methasulfocarb preparations, oxyethylene docosanol preparations, ethephon preparations, cloxyfonac preparations, gibberellin, streptomycin preparations, daminozide preparations, 4-CPA preparations, ancymidol preparations, inabenfide preparations, uniconazole preparations, chlormequat preparations, dikegulac preparations, daminozide preparations, mefluidide preparations, calcium carbonate preparations and piperonyl butoxide preparations. Among them, brassinolides, cholines, isoprothiolane preparations, and hydroxyisoxasole preparations are preferred.

Examples of the sugars usable here include glucose, sucrose, xylitol, sorbitol, galactose, xylose, mannose, arabinose, madulose, ribose, rhamnose, fructose, maltose, lactose and maltotriose. Among them, glucose, sucrose, and galactose are preferred.

Examples of the amino acids usable here include asparagine, glutamine, histidine, tyrosine, glycine, arginine, alanine, tryptophan, methionine, valine, proline, leucine, lysine and isoleucine.

Examples of the organic acids usable here include formic acid, acetic acid, phosphoric acid, propionic acid, butyric acid, valeric acid, phthalic acid, benzoic acid, lactic acid, citric acid, tartaric acid, malonic acid, malic acid, succinic acid, glycolic acid, glutamic acid, aspartic acid, maleic acid, caproic acid, caprylic acid, myristic acid, stearic acid, palmitic acid, pyruvic acid, α-ketoglutaric acid and levulinic acid. Among them, acetic acid, propionic acid, malic acid, succinic acid, glutamic acid, and levulinic acid are preferred.

Examples of the alcohols usable herein include methanol, ethanol, propanol, butanol, pentanol, hexanol and glycerol with methanol and ethanol being preferred.

Examples of the vitamins usable herein include nicotinic acid amide, vitamin B6, vitamin B12, vitamin B5, vitamin C, vitamin B13, vitamin B1, vitamin B3, vitamin B2, vitamin K3, vitamin A, vitamin D2, vitamin D3, vitamin K1, α-tocopherol, β-tocopherol, γ-tocopherol, δ-tocopherol, p-hydroxybenzoic acid, biotin, folic acid, nicotinic acid, pantothenic acid and α-liponic acid.

Examples of the minerals usable here include nitrogen, phosphorus, potassium, boron, manganese, zinc, copper, iron, molybdenum and magnesium.

The composition to be used according to the present invention may be in the form of, for example, powder, granules or liquid. These formulations may be produced by a conventional method with the use of, for example, solvents, dispersion media or extenders.

The substance to be used in the composition and the method the present invention may be in the form of either a foliage treatment substance or a soil treatment substance. Alternately, it may be absorbed by plants before planting or cutting (i.e., a soaking treatment agent). Alternately, it may be added to water for hydroponic use.

Although each of these treatments may be performed at any stage of the growth of a plant, it is particularly effective to perform the treatment at the seedling stage or the grain maturing stage. A single application can achieve satisfactory results. However the results can be further improved by repeating the application. When the application is to be performed twice or more, the treatment methods as described above may be appropriately combined with each other, if required. When the composition of the invention is used together with other chemicals or fertilizers in order to facilitate the application, it may be mixed with any materials so long as the effects thereof are not deteriorated thereby.

The compositions used according to the invention preferably consist essentially of an effective amount of an oxalate and more preferably consist of an effective amount of an oxalate, at least one dispersant, at least one carrier, and, optionally, water. The compositions may in one embodiment contain the oxalate and water with no carrier. The compositions of the invention may consist essentially of an oxalate compound and water. The compositions may also consist of an oxalate compound and water.

Among the many optional additives suitable for use in compositions of the invention include surfactants and other ingredients, such as dispersants, stickers, antifoam agents, antifreezing agents, dyestuffs, thickeners, adhesives, protective colloids, penetrating agents, stabilizing agents, sequestering agents, antiflocculating agents, corrosion inhibitors, and polymers.

More generally, the compositions of the invention can include all kinds of solid or liquid additives which are known in the art of crop protection and horticultural pest control treatments.

The surfactants can be of the emulsifying or wetting type and can be ionic or non-ionic. Possible surfactants are salts of polyacrylic or lignosulfonic acids; salts of phenolsulfonic or naphthalenesulfonic acids; polycondensates of ethylene oxide with fatty alcohols or fatty acids or fatty amines or substituted phenols (particularly alkylphenols or arylphenols); ester-salts of sulfosuccinic acids; taurine derivatives, such as alkyl taurates; phosphoric esters; or esters of alcohols or polyoxyethylated phenols. When the spraying vehicle is water, the use of at least one surfactant is generally required because the active ingredients are not water-soluble.

Dusting powders, granulates, solution, emulsifiable concentrates, emulsions, suspended concentrates and aerosols are also contemplated within the invention. The wettable powders according to the invention can be prepared in such a way that they contain from 1% to 95% by weight of the active material, and they normally contain, in addition to a solid support, from 0 to 5% by weight of a wetting agent, from 3 to 10% by weight of a dispersant, and, when necessary, from 0 to 10% by weight of one or more stabilizers and/or other additives, such as penetration agents, adhesives or anti-clumping agents, or colorants. The compositions according to the invention can contain other ingredients, for example protective colloids, adhesives or thickeners, thixotropic agents, stabilizers or sequestrants, as well as other active materials known to have pesticidal properties, especially certain fungicides, acaricides, and insecticides.

The present invention can be practiced with all turfgrasses, including cool season turfgrasses and warm season turfgrasses. Examples of cool season turfgrasses are bluegrasses (Poa spp.), such as Kentucky bluegrass (Poa pratensis L.), rough bluegrass (Poa trivialis L.), Canada bluegrass (Poa compressa L.), annual bluegrass (Poa annua L.), upland bluegrass (Poa glaucantha Gaudin), wood bluegrass (Poa nemoralis L.), and bulbous bluegrass (Poa bulbosa L.); the bentgrasses and redtop (Agrostis spp.), such as creeping bentgrass (Agrostis palustris Huds.), colonial bentgrass (Agrostis tenuis Sibth.), velvet bentgrass (Agrostis canina L.), South German Mixed Bentgrass (Agrostis spp. including Agrostis tenius Sibth., Agrostis canina L., and Agrostis palustris Huds.), and redtop (Agrostis alba L.); the fescues (Festucu spp.), such as red fescue (Festuca rubra L. spp. rubra), creeping fescue (Festuca rubra L.), chewings fescue (Festuca rubra commutata Gaud.), sheep fescue (Festuca ovina L.), hard fescue (Festuca longifolia Thuill.), hair fescue (Festucu capillata Lam.), tall fescue (Festuca arundinacea Schreb.), meadow fescue (Festuca elanor L.); the ryegrasses (Lolium spp.), such as annual ryegrass (Lolium multiflorum Lam.), perennial ryegrass (Lolium perenne L.), italian ryegrass (Lolium multiflorum Lam.); and the wheatgrasses (Agropyron spp.), such as fairway wheatgrass (Agropyron cristatum (L.)Gaertn.), crested wheatgrass (Agropyron desertorum (Fisch.)Schult.), and western wheatgrass (Agropyron smithii Rydb.). Other cool season turfgrasses include beachgrass (Ammophila breviligulata Fern.), smooth bromegrass (Bromus inermis Leyss.), cattails such as Timothy (Phleum pratense L.), sand cattail (Phleum subulatum L.), orchardgrass (Dactylis glomerata L.), weeping alkaligrass (Puccinellia distans (L.)Parl.) and crested dog's-tail (Cynosurus cristatus L.). Examples of warm season turfgrasses include Bermudagrass (Cynodon spp. L. C. Rich), zoysiagrass (Zoysia spp. Wilid.), St. Augustine grass (Stenotaphrum secundatum Walt Kuntze), centipedegrass (Eremochloa ophiuroides Munro Hack.), carpetgrass (Axonopus affinis Chase), Bahia grass (Paspalum notatum Flugge), Kikuyugrass (Pennisetum clandestinum Hochst. ex Chiov.), buffalo grass (Buchloe dactyloids (Nutt.)Engelm.), Blue gramma (Bouteloua gracilis (H.B.K.) Lag. ex Griffiths), seashore paspalum (Paspalum vaginatum Swartz) and sideoats grama (Bouteloua curtipendula (Michx. Torr.). In one aspect of the invention, cool season turfgrasses are generally preferred for treatment according to the invention. In another aspect of the invention, warm season turfgrasses are also preferred for treatment according to the invention. More preferred is bluegrass, bentgrass and redtop, fescue, and ryegrass. Bentgrass is most preferred.

Other plants to be treated with the composition of the invention are not particularly restricted. Examples thereof include cereals such as rice, barley, wheat, corn, barnyard millet and foxtail millet; vegetables such as pumpkin, turnip, cabbage, radish, Chinese cabbage, spinach, pimento and tomato; fruit trees such as orange, apple, persimmon, Japanese apricot, pear, grape and peach; flowers such as roses, chrysanthemum, Transvaal daisy, pansy, orchid, peony and tulip; trees such as azalea, oak (Quercus acutissima), Japanese cedar, white cedar, Japanese oak and beech; beans such as adzuki bean, kidney bean, soybean, peanut, broad bean and pea; turfgrass, forage grass; bent grass and field grass; potatoes such as potato, sweet potato, Japanese taro, yam and taro; onions such as Welsh onion, onion and scallion; and pasture grasses such as alfalfa, clover and Chinese milk vetch.

Other embodiments are encompassed by this inventions. In one embodiment, the invention is directed to a method of killing insects on plant tissue which comprises: applying a solution that includes a substance wherein the substance is a substrate for a hydrogen-peroxide generating enzyme. In a preferred embodiment, the substrate is an oxalic acid, glucose, citrate or the like. In a preferred embodiment, the method further comprises applying hydrogen peroxide to plant tissue. In a preferred embodiment, the hydrogen peroxide in the solution having a concentration of between 0.05 to 3.00% by weight. In a preferred embodiment, the hydrogen peroxide in the solution has a concentration of between 0.05 and 1.5% by weight. In a preferred embodiment, the method further includes at least one step of reapplying the solution.

In another embodiment, the invention is directed to a method for killing insects on plant tissue which comprises: applying a solution that includes a substance wherein the substance is a substrate for a hydrogen-peroxide generating enzyme and the solution further includes an acid selected from the group consisting of acetic acid and phosphoric acid. In a preferred embodiment, the substrate is an oxalic acid, glucose, citrate or the like.

In a preferred embodiment, the method further comprises applying hydrogen peroxide to plant tissue, said hydrogen peroxide in the solution has a concentration of between 0.05 and 1.5% by weight and the solution further includes an acid selected from the group consisting of acetic acid and phosphoric acid.

In a preferred embodiment, the solution is applied by hydraulic spraying.

In a preferred embodiment, the solution is applied by misting.

In a preferred embodiment, the solution is applied by fogging.

In a preferred embodiment, the solution is applied by injecting the solution into a commercial cooling system.

In a preferred embodiment, the solution is injected into a recirculating subirrigation nutrient system solution to control microbial growth in the irrigation water.

In a preferred embodiment, the solution is applied to the plant tissue at a variety of stages in the plant production cycle.

In a preferred embodiment, the solution is injected into the water that is misted over terminal stem cuttings until they reach root sufficiency.

In a preferred embodiment, the step of applying said solution to plant tissue is repeated a plurality of times.

In a preferred embodiment, the method further includes at least one step of reapplying the solution.

In a preferred embodiment, a method of killing insects on plant tissue which comprises: applying a solution that includes a substance wherein the substance is a substrate for a hydrogen-peroxide generating enzyme, wherein the substrate is an oxalic acid, glucose, citrate or a like. In a more preferred embodiment, the method further comprises applying hydrogen peroxide to plant tissue, wherein the hydrogen peroxide in the solution is present at a concentration of between 0.05 to 3.00% by weight.

In a preferred embodiment, a method for killing insects on plant tissue which comprises: applying a solution that includes a substance where in the substance is a substrate for a hydrogen-peroxide generating enzyme and the solution further includes an acid selected from the group consisting of acetic acid and phosphoric acid, wherein the substrate is an oxalic acid, glucose, citrate or a like. In a more preferred embodiment, the method further comprises hydrogen peroxide to plant tissue, said hydrogen peroxide in the solution has a concentration of between 0.05 and 1.5% by weight and the solution further includes an acid selected from the group consisting of acetic acid and phosphoric acid.

In a preferred embodiment, a method of killing or controlling the larvae or grub of insects which comprise the southern masked chafer, the northern masked chafer, the Japanese beetle, the European chafer, the cupreous chafer, the oriental beetle and the May or June beetle which comprises contacting the larvae or grub in the soil and/or on the plant with an insecticidally effective amount of a susbstance wherein the substance is a substrate for a hydrogen peroxide generating enzyme. In a preferred embodiment, the method further comprises applying hydrogen peroxide to plant tissue. In a preferred embodiment, the hydrogen peroxide in the solution having a concentration of between 0.05 to 3.00% by weight.

In a more preferred embodiment, the substrate is an oxalic acid, glucose, citrate or the like. In a most preferred embodiment, the substrate is an oxalic acid.

In general, the rate of application at the locus is from 0.001 to 100 kilograms of oxalate per hectare (kg/ha), most preferably from about 0.01 to about 10 kg/ha, more preferably from about 0.1 to about 1 kg/ha, most preferably from about 0.2 to about 0.8 kg/ha. The compositions of the invention are applied by known methods.

In one embodiment of this invention, the insect is the Japanese beetle.

In one embodiment of this invention, the substrate is applied to an area where turfgrass, ornamental plants or food crops are grown or will be grown. In one preferred embodiment, the substrate is applied to turfgrass.

In one embodiment of this invention, the substrate is applied as a constituent of a composition comprising an insecticidally effective amount of the compound and an agronomically acceptable carrier.

In one embodiment of this invention, is present at from about 0.0001 to about 99% by weight of the composition.

In one embodiment of this invention, the agronomically acceptable carrier is a solid.

In one embodiment of this invention, this invention is directed to a method of controlling the larvae or grub of insects which comprise the southern masked chafer, the northern masked chafer, the Japanese beetle, the European chafer, the cupreous chafer, the oriental beetle and the May or June beetle which comprises contacting the larvae or grub in the soil or on the plant with an insecticidally effective amount of a substance wherein the substance is a substrate for a hydrogen peroxide generating enzyme, wherein the method further comprises applying hydrogen peroxide to plant tissue, wherein the hydrogen peroxide in the solution having a concentration of between 0.05 to 3.00% by weight.

In one embodiment of this invention, this invention is directed to a method of controlling the larvae or grub of insects which comprise the southern masked chafer, the northern masked chafer, the Japanese beetle, the European chafer, the cupreous chafer, the oriental beetle and the May or June beetle which comprises contacting the larvae or grub in the soil or on the plant with an insecticidally effective amount of a substance wherein the substance is a substrate for a hydrogen peroxide generating enzyme, wherein the method further includes an acid selected from the group consisting of acetic acid and phosphoric acid.

In one embodiment of this invention, this invention is directed to a method of controlling the larvae or grub of insects which comprise the southern masked chafer, the northern masked chafer, the Japanese beetle, the European chafer, the cupreous chafer, the oriental beetle and the May or June beetle which comprises contacting the larvae or grub in the soil or on the plant with an insecticidally effective amount of a substance wherein the substance is a substrate for a hydrogen peroxide generating enzyme, wherein the substrate is an oxalic acid, glucose, citrate or a like.

In one embodiment of this invention, this invention is directed to a method of controlling the larvae or grub of insects which comprise the southern masked chafer, the northern masked chafer, the Japanese beetle, the European chafer, the cupreous chafer, the oriental beetle and the May or June beetle which comprises contacting the larvae or grub in the soil or on the plant with an insecticidally effective amount of a substance wherein the substance is a substrate for a hydrogen peroxide generating enzyme, wherein the substrate is an oxalic acid, glucose, citrate or the like, wherein the method further comprises hydrogen peroxide to plant tissue.

In one embodiment of this invention, this invention is directed to a method of controlling the larvae or grub of insects which comprise the southern masked chafer, the northern masked chafer, the Japanese beetle, the European chafer, the cupreous chafer, the oriental beetle and the May or June beetle which comprises contacting the larvae or grub in the soil or on the plant with an insecticidally effective amount of a substance wherein the substance is a substrate for a hydrogen peroxide generating enzyme, wherein the substrate is an oxalic acid, glucose, citrate or a like, wherein the method further comprises hydrogen peroxide to plant tissue, said hydrogen peroxide in the solution has a concentration of between 0.05 and 3.0% by weight and the solution further includes an acid selected from the group consisting of acetic acid and phosphoric acid.

In one embodiment, the invention is directed to a pesticidal composition which comprises, in variable quantities, hydrogen peroxide generating substance and a pesticidally active compound in free form or in the form of an agrochemically acceptable salt thereof.

In one embodiment, the invention is directed to a pesticidal composition which comprises, in variable quantities, hydrogen peroxide generating substance and a pesticidally active compound in free form or in the form of an agrochemically acceptable salt thereof wherein the hydrogen peroxide generating substance is an oxalate, citrate, glucose or the like.

In one embodiment, the invention is directed to a pesticidal composition which comprises, in variable quantities, hydrogen peroxide generating substance and a pesticidally active compound in free form or in the form of an agrochemically acceptable salt thereof wherein the composition is further comprising a a chemical inducer which induces systemic acquired resistance, 5-aminolevulinic acid or a salt thereof, acetosyringone, Thidiazuron (TDZ), Brassinolide, 6-Benzylaminopurine (6-BA), Kinetin, and/or zeatin or the like.

In one embodiment, the invention is directed to a pesticidal composition which comprises, in variable quantities, an oxalate and a pesticidally active compound in free form or in the form of an agrochemically acceptable salt thereof, and at least one auxiliary or carrier material. In a more preferred embodiment, the pesticidal composition may be used for treating infectious or pathogenic pest in a plant comprising: a pesticidal composition including an effective amount of at least one effective pesticidal form of oxalate and at least one pesticide, wherein said effective amount is a lethal dosage of oxalate and wherein said pesticide composition is adapted to be administered to a plant on a periodic basis in a lethal dosage to a pest. In a more preferred embodiment, the pesticidal composition wherein the effective pesticidal form of at least one of oxalate is selected from the group of oxalic acid in a free acid, ester, lactone and salt form. In a more preferred embodiment, the pesticidal composition wherein the effective pesticidal form of oxalate is selected from the group of fungi, natural foods, for processed foods, beverages, liquids, and juices, containing at least one of oxalic acid and oxalate. In a more preferred embodiment, the invention is directed to the pesticidal composition wherein the composition is at least a therapeutic quantity of oxalate from a natural source. In a more preferred embodiment, the invention is directed to the pesticidal composition, wherein the composition is oxalic acid dihydrate and the at least one carrier and/or diluent. In a more preferred embodiment, the invention is directed to the pesticidal composition comprises at least one of carrier and/or diluent In a more preferred embodiment, the invention is directed to a method of producing the therapeutic composition comprising the steps of mixing a dilute concentration of at least one therapeutically effective form of oxalate with a solvent.

In a more preferred embodiment, the invention is directed to the method, wherein said therapeutically effective biocidal form of at least one of oxalic acid and oxalate is selected from the group of oxalic acid in a free acid, ester, lactone or salt form.

In a more preferred embodiment, the invention is directed to the pesticidal composition wherein the pesticide further comprises a carrier.

In a more preferred embodiment, the invention is directed to the pesticidal composition wherein the carrier is selected from the group consisting of: water, stabilizers, emulsifiers, oils, surfactants, antioxidants and UV screens.

In a more preferred embodiment, the invention is directed to the pesticidal composition wherein the surfactant is a non-ionic surfactant.

In a more preferred embodiment, the invention is directed to the pesticidal composition wherein the surfactant is a non-ionic organic surfactant.

In a more preferred embodiment, the invention is directed to the pesticidal composition wherein the surfactant is nonylphenoxy polyethoxyethanol.

In a more preferred embodiment, the invention is directed to the pesticidal composition wherein the pesticide comprises 0.01 to 0.1% surfactant.

In a more preferred embodiment, the invention is directed to the pesticidal composition wherein the pesticide comprises about 0.03% (w/v) surfactant.

In a more preferred embodiment, the invention is directed to the pesticidal composition wherein the plant is selected the group consisting of an ornamental plant, a fruit plant, an herb or a medicinal plant, a fruit plant, carnation, rose, lavender, tulip, delphinium, dahlia, citrus fruit tree, grape vine, strawberry, coffee, banana, tomato, rockmelon, watermelon plant, red clover, a vegetable plant, chamomile, aloe, evening primrose, skullcap, Echinacca, saw palmetto, feverfew, witch hazel, valerian, tea tree, garlic, eucalyptus, basil, parsley or ginseng plant.

In a more preferred embodiment, the invention is directed to the pesticidal composition wherein the vegetable plant is a bean, corn, carrot, cucumber, potato, radish, spinach, pea, lettuce, onion, cabbage, broccoli, cauliflower, zucchini or turnip plant.

In a more preferred embodiment, the invention is directed to the pesticidal composition wherein the plant is selected from the group consisting of a cereal plant, a turf grass plant, and a forage grass plant.

A method of improving turfgrass quality comprising applying an effective amount of a composition containing a substrate for a hydrogen peroxide generating enzyme to the turfgrass plants. In a more preferred embodiment, the substrate is selected from the group consisting of oxalate, glucose and citrate and alike. In a more preferred embodiment, the method of improving turfgrass quality comprises 0.001 to 100 kg per hectare of the substrate is applied. In a more preferred embodiment, about 0.01 to about 10 kg per hectare of the substrate is applied.

In one of embodiment, the invention is directed to a method of improving turfgrass quality comprising applying to the turfgrass an effective amount of a composition consisting essentially of an oxalate at a rate such that about 0.01 to about 10 kg per hectare of the oxalate is applied.

In one of embodiment, the invention is directed to a method of improving turfgrass quality comprising applying to the turfgrass an effective amount of a composition consisting of an oxalate, at least one dispersant, and at least one carrier at a rate such that about 0.01 to about 10 kg per hectare of the oxalate is applied.

In one of embodiment, the invention is directed to a method of improving turfgrass quality comprising applying an effective amount of a composition consisting of an oxalate and water to the turfgrass at a rate such that about 0.01 to about 10 kg per hectare of the oxalate is applied.

In one of embodiment, the invention is directed to a composition for improving turfgrass consisting essentially of an effective turfgrass-improving amount of an oxalate, at least one dispersant acceptable for use in turfgrass, at least one carrier acceptable for use in turfgrass, and optionally water.

In one of embodiment, the invention is directed to a composition for improving turfgrass consisting of an effective turfgrass-improving amount of a composition containing a substance wherein the substance is a substrate for a hydrogen peroxide generating enzyme, at least one dispersant acceptable for use in turfgrass, at least one carrier acceptable for use in turfgrass, and optionally water. In a preferred embodiment, the invention is directed to a composition for improving turfgrass consisting of an effective turfgrass-improving amount of a composition containing a substance wherein the substance is a substrate for a hydrogen peroxide generating enzyme, at least one dispersant acceptable for use in turfgrass, at least one carrier acceptable for use in turfgrass, and optionally water, wherein the substrate is selected from the group consisting of oxalate, glucose and citrate or a like. In a preferred embodiment, the invention is directed to a composition for improving turfgrass consisting of an effective turfgrass-improving amount of a composition containing an oxalate, at least one dispersant acceptable for use in turfgrass, at least one carrier acceptable for use in turfgrass, and optionally water.

In one of embodiment, the invention is directed to a composition for improving turfgrass consisting of an effective turfgrass-improving amount of containing a substance and water. In one of embodiment, the invention is directed to the substrate is a substrate for a hydrogen peroxide generating enzyme. In one of embodiment the substrate is selected from the group consisting of oxalate, glucose and citrate or the like. In one of embodiment, the composition containing an oxalate Only.

In one of embodiment, the invention is directed to a wettable powder composition for improving turfgrass comprising from about 1% to 95% by weight of a substance on a solid support, from about 0 to 5% by weight of a wetting agent, from about 3 to 10% by weight of a dispersant, and optionally from about 0 to 10% by weight of one or more stabilizers and/or additives, wherein the substance is a substrate for a hydrogen peroxide generating enzyme In one of embodiment, the substrate is selected from the group consisting of oxalate, glucose and citrate or the like. In one of embodiment, the invention is directed to a composition containing an oxalate. In one of embodiment, the invention is directed to a wettable powder composition additionally comprising one or more fungicides, nematicides, acaricides, and/or insecticides.

In one embodiment, this invention can be practiced by treating the plants according to this invention with oxalate alone and/or in combination of 5-aminolevulinic acid or a salt thereof (U.S. Pat. No. 5,298,482—the entire content of this patent is also incorporated by reference) in order to improve the growth and the performance of the plant as well as to increase killing insects, nematodes, fungi and/or other pathogens. In one embodiment, the invention is directed to a method for promoting the growth of a plant which comprises treating said plant with oxalate and 5-aminolevulinic acid or a salt thereof wherein the 5-aminolevulinic acid or a salt thereof is used at 1 to 1,000 ppm and 10 to 1,000 1/10 a, in the case of foliage treatment, 1 to 1,000 g/10 a, in the case of soil treatment, or 0.001 to 10 ppm, in the case of soaking treatment, of 5-aminolevulinic acid or a salt thereof wherein the method further comprises a substance wherein the substance is a substrate for hydrogen peroxide generating enzyme. In one of embodiment, the hydrogen peroxide generating substrate is an oxalate, glucose, citrate or the like.

In one embodiment, the invention is directed to a method for promoting the rooting of a plant which comprises treating said plant with oxalate and 5-aminolevulinic acid or a salt thereof wherein the 5-aminolevulinic acid or a salt thereof is used at 1 to 1,000 ppm and 10 to 1,000 1/10 a, in the case of foliage treatment, 1 to 1,000 g/10 a, in the case of soil treatment or 0.001 to 10 ppm, in the case of soaking treatment, from 1 hour to 1 week of 5-aminolevulinic acid or a salt thereof wherein the method further comprises a substance wherein the substance is a substrate for hydrogen peroxide generating enzyme. In one embodiment, the hydrogen peroxide generating substrate is an oxalate, glucose, citrate or the like.

In one embodiment, the invention is directed to a method for promoting the rooting ratio of a plant which comprises treating said plant with oxalate and 5-aminolevulinic acid or a salt thereof wherein the 5-aminolevulinic acid or a salt thereof is used at 1 to 1,000 ppm and 10 to 1,000 1/10 a, in the case of foliage treatment, 1 to 1,000 g/10 a, in the case of soil treatment or 0.001 to 10 ppm, in the case of soaking treatment, from 1 hour to 1 week, of 5-aminolevulinic acid or a salt thereof wherein the method further comprises a substance wherein the substance is a substrate for s hydrogen peroxide generating enzyme. In one embodiment, the hydrogen peroxide generating substrate an oxalate, glucose, citrate or the like.

In one embodiment, the invention is directed to a method for growing good seedlings of a plant which comprises treating said seedlings or part thereof with oxalate and 5-aminolevulinic acid or a salt thereof wherein the 5-aminolevulinic acid or a salt thereof is used at 1 to 1,000-ppm and 10 to 1,000 1/10 a, in the case of foliage treatment, 1 to 1,000 g/10 a, in the case of soil treatment, or 0.001 to 10 ppm, in the case of soaking treatment, from 1 hour to 1 week, of 5-aminolevulinic acid or a salt thereof wherein the method further comprises a substance wherein the substance is a substrate for a hydrogen peroxide generating enzyme. In one embodiment, the hydrogen peroxide generating substrate is an oxalate, glucose, citrate or the like.

In one embodiment, the invention is directed to a method for reducing lodging of a plant which comprises treating said plant or part thereof with 1 to 1,000 ppm and 10 to 1,000 1/10 a, in the case of foliage treatment, 1 to 1,000 g/10 a, in the case of soil treatment, or 0.001 to 10 ppm in the case of soaking treatment, from 1 hour to 1 week, of 5-aminolevulinic acid or a salt thereof wherein the method further comprises a substance wherein the substance is a substrate for a hydrogen peroxide generating enzyme. In one embodiment, the hydrogen peroxide generating substrate is an oxalate, glucose, citrate or the like.

In one embodiment, the invention is directed to a method for increasing the yield of a plant which comprises treating said plant or part thereof with 1 to 1,000 ppm and 10 to 1,0001/10 a, in the case of foliage treatment, 1 to 1,000 g/10 a, in the case of soil treatment, or 0.001 to 10 ppm in the case of soaking treatment, from 1 hour to 1 week, of 5-aminolevulinic acid or a salt thereof wherein the method further comprises a substance wherein the substance is a substrate for s hydrogen peroxide generating enzyme. In one embodiment, the hydrogen peroxide generating substrate is an oxalate, glucose, citrate or the like.

In one embodiment, the invention is directed to a method for improving the cold resistance of a plant which comprises treating said plant or part thereof with oxalate and 5-aminolevulinic acid or a salt thereof wherein the 5-aminolevulinic acid or a salt thereof is used at 1 to 1,000 ppm and 10 to 1,000 1/10 a, in the case of foliage treatment, 1 to 1,000 g/10 a, in the case of soil treatment, or 0.001 to 10 ppm in the case of soaking treatment, from 1 hour to 1 week, of 5-aminolevulinic acid or a salt thereof wherein the method further comprises a substance wherein the substance is a substrate for s hydrogen peroxide generating enzyme. In one embodiment, the hydrogen peroxide generating substrate is an oxalate, glucose, citrate or the like.

In one embodiment, the invention is directed to a method for maintaining the freshness of a plant which comprises treating said plant with oxalate and 5-aminolevulinic acid or a salt thereof wherein the 5-aminolevulinic acid or a salt thereof is used at 1 to 1,000 ppm and 10 to 1,0001/10 a, in the case of foliage treatment, or 1 to 27 1,000 g/10 a, in the case of soil treatment, of 5-aminolevulinic acid or a salt thereof wherein the method further comprises a substance wherein the substance is a substrate for a hydrogen peroxide generating enzyme. In one embodiment, the hydrogen peroxide generating substrate is an oxalate, glucose, citrate or the like.

In one embodiment, the invention is directed to a method for maintaining or improving the green color of a plant which comprises treating said plant with 1 to oxalate and 5-aminolevulinic acid or a salt thereof wherein the 5-aminolevulinic acid or a salt thereof is used at 1,000 ppm and 10 to 1,000 1/10 a, in the case of foliage treatment, 1 to 1,000 g/10 a, in the case of soil treatment, or 0.001 to 10 ppm, in the case of soaking treatment, from 1 hour to 1 week, of 5-aminolevulinic acid or a salt thereof wherein the method further comprises a substance wherein the substance is a substrate for a hydrogen peroxide generating enzyme. In one embodiment, the hydrogen peroxide generating substrate is an oxalate, glucose, citrate or the like.

In one embodiment, the invention is directed to a method for relieving chemical damage to a plant which comprises treating said plant with oxalate and 5-aminolevulinic acid or a salt thereof wherein the 5-aminolevulinic acid or a salt thereof is used at 1 to 1,000 ppm and 10 to 1,0001/10 a, in the case of foliage treatment, 1 to 1,000 g/10 a, in the case of soil treatment, or 0.001 to 10 ppm, in the case of soaking treatment, from 1 hour to 1 week, of 5-aminolevulinic acid or a salt thereof wherein the method further comprises a substance wherein the substance is a substrate for a hydrogen peroxide generating enzyme. In one embodiment, the hydrogen peroxide generating substrate is an oxalate, glucose, citrate or the like.

In one embodiment, the invention is directed to a method for increasing the number of tillers of a plant which comprises treating said plant with oxalate and 5-aminolevulinic acid or a salt thereof wherein the 5-aminolevulinic acid or a salt thereof is used at 1 to 1,000 ppm and 10 to 1,000 1/10 a, in the case of foliage treatment, 1 to 1,000 g/10 a, in the case of soil treatment, or 0.001 to 10 ppm, in the case of soaking treatment, from 1 hour to 1 week, of 5-aminolevulinic acid or a salt thereof wherein the method further comprises a substance wherein the substance is a substrate for a hydrogen peroxide generating enzyme. In one embodiment, the hydrogen peroxide generating substrate is an oxalate, glucose, citrate or the like.

In one embodiment, the invention is directed to a method for shortening the time required for the growth of a plant which comprises treating said plant with oxalate and 5-aminolevulinic acid or a salt thereof wherein the 5-aminolevulinic acid or a salt thereof is used at 1 to 1,000 ppm and 10 to 1,000 1/10 a, in the case of foliage treatment, 1 to 1,000 g/10 a, in the case of soil treatment, or 0.001 to 10 ppm, in the case of soaking treatment, from 1 hour to 1 week, of 5-aminolevulinic acid or a salt thereof wherein the method further comprises a substance wherein the substance is a substrate for a hydrogen peroxide generating enzyme. In one embodiment, the hydrogen peroxide generating substrate is an oxalate, glucose, citrate or the like.

In one embodiment, the invention is directed to a method for promoting the growth of an organ of a plant which comprises incubating calluses, shoot primordia or hairly roots of said plant in a medium containing 0.001 to 10 ppm of 5-aminolevulinic acid or a salt thereof for 1 hour to 1 week wherein the method further comprises a substance wherein the substance is a substrate for a hydrogen peroxide generating enzyme. In one embodiment, the hydrogen peroxide generating substrate is an oxalate, glucose, citrate or the like.

In one embodiment, the invention is directed to a method for enhancing the photosynthetic activity of a plant which comprises treating said plant with oxalate and 5-aminolevulinic acid or a salt thereof wherein the 5-aminolevulinic acid or a salt thereof is used at 1 to 1,000 ppm and 10 to 1,000 1/10 a, in the case of foliage treatment, 1 to 1,000 g/10 a, in the case of soil treatment, or 0.001 to 10 ppm, in the case of soaking treatment, from 1 hour to 1 week, of 5-aminolevulinic acid or a salt thereof wherein the method further comprises a substance wherein the substance is a substrate for a hydrogen peroxide generating enzyme. In one embodiment, the hydrogen peroxide generating substrate is an oxalate, glucose, citrate or the like.

In one embodiment, the invention is directed to a method for suppressing the respiration of a plant which comprises treating said plant with oxalate and 5-aminolevulinic acid or a salt thereof wherein the 5-aminolevulinic acid or a salt thereof is used at 1 to 1,000 ppm and 10 to 1,0001/10 a, in the case of foliage treatment, 1 to 1,000 g/10 a, in the case of soil treatment, or 0.001 to 10 ppm, in the case of soaking treatment, from 1 hour to 1 week, of 5-aminolevulinic acid or a salt thereof wherein the method further comprises a substance wherein the substance is a substrate for a hydrogen peroxide generating enzyme. In one embodiment, the hydrogen peroxide generating substrate an oxalate, glucose, citrate or the like.

In one embodiment, the invention is directed to a method for enhancing the ability to absorb CO₂ of a plant which comprises treating said plant with oxalate and 5-aminolevulinic acid or a salt thereof wherein the 5-aminolevulinic acid or a salt thereof is used at 1 to 1,000 ppm and 10 to 1,000 1/10 a, in the case of foliage treatment, 1 to 1,000 g/10 a, in the case of soil treatment, or 0.001 to 10 ppm, in the case of soaking treatment, from 1 hour to 1 week, of 5-aminolevulinic acid or a salt thereof wherein the method further comprises a substance wherein the substance is a substrate for a hydrogen peroxide generating enzyme. In one embodiment, the hydrogen peroxide generating substrate is an oxalate, glucose, citrate or the like.

In one embodiment, the invention is directed to a method for increasing the chlorophyll content of a plant which comprises treating said plant with oxalate and 5-aminolevulinic acid or a salt thereof wherein the 5-aminolevulinic acid or a salt thereof is used at 1 to 1,000 ppm and 10 to 1,000 1/10 a, in the case of foliage treatment, 1 to 1,000 g/10 a, in the case of soil treatment, or 0.001 to 10 ppm, in the case of soaking treatment, from 1 hour to 1 week, of 5-aminolevulinic acid or a salt thereof wherein the method further comprises a substance wherein the substance is a substrate for a hydrogen peroxide generating enzyme. In one embodiment, the hydrogen peroxide generating substrate is an oxalate, glucose, citrate or the like.

In one preferred embodiment, the invention is directed to a method wherein the foliage treatment is performed with 10 to 500 ppm of 5-aminolevulinic acid or a salt thereof at 50 to 300 1/10 a, the soil treatment is performed with 10 to 500 g/10 a of 5-aminolevulinic acid or a salt thereof, or the soaking treatment is performed with 0.01 to 5 ppm of 5-aminolevulinic acid or a salt thereof for 3 hours to 1 day wherein the method is further comprised of an oxalate or other SAR induces which are encompassed by this invention.

In one preferred embodiment, the salt of 5-aminolevulinic acid comprises one or more compounds selected from among the acid-addition salts hydrochloride, phosphate, nitrate, sulfate, acetate, propionate, butyrate, valerate, citrate, fumarate, maleate or malate and the metal salts sodium, potassium or calcium.

In one preferred embodiment for promoting the growth of a plant which comprises treating said plant with one or more compounds selected from other plant growth regulators, sugars, amino acids, organic acids, alcohols, vitamins or minerals in combination with said 5-aminolevulinic acid or a salt thereof. In one preferred embodiment the plant growth regulator is epibrassinolide or choline chloride. In one preferred embodiment the sugar is glucose or sucrose.

In one preferred embodiment the amino acid is selected from the group consisting of asparagine, glutamine, histidine, tyrosine, glycine, arginine, alanine, tryptophan, methionine, valine, proline, leucine, lysine and isoleucine.

In one preferred embodiment the organic acid is selected from among formic acid, acetic acid, propionic acid, butyric acid, valeric acid, oxalic acid, phthalic acid, benzoic acid, lactic acid, citric acid, tartaric acid, malonic acid, malic acid, succinic acid, glycolic acid, glutamic acid, aspartic acid, maleic acid, caproic acid, caprylic acid, myristic acid, stearic acid, palmitic acid, pyruvic acid, α-ketoglutaric acid and levulinic acid.

In one preferred embodiment the composition comprises a vitamin which is selected from the group consisting of nicotinic acid amide, vitamin B₆, vitamin B12, vitamin B₅, vitamin C, vitamin B13, vitamin B₁, vitamin B₃, vitamin B₂, vitamin K₃, vitamin A, vitamin D₂, vitamin D₃, vitamin K₁, α-tocopherol, β-tocopherol, γ-tocopherol, δ-tocopherol, p-hydroxybenzoic acid, biotin, folic acid, nicotinic acid, pantothenic acid and α-liponic acid.

Although oxalic acid has proven most effective in improving the performance of the compositions of the invention, other components are also effective in formulating the compositions. Organic acids, particularly polycarboxylic acids, may be added to the compositions to enhance herbicidal effectiveness.

Preferred polycarboxylic acids include dicarboxylic acids, Suitable dicarboxylic acids that may be added to the formulations include oxalic acid, malonic acid, succinic acid, glutaric acid, maleic acid, adipic acid, and fumaric acid, salts thereof and mixtures thereof, with oxalic acid being preferred: Suitable salts include, for example, alkali metal salts such as sodium and potassium salts, alkanolamine salts and alkylamine salts. Preferred salts include potassium oxalate, dipotassium oxalate, sodium oxalate, disodium oxalate, diammonium oxalate, diethanolamine oxalate, dimethylamine oxalate, alkanolamine salts of oxalic acid, and lower alkylamine salts of oxalic acid. Formulations contain such compounds in an amount sufficient to enhance the resulting efficacy of the formulation.

The aforementioned compositions can be used to treat diseases or infestations caused by nematodes of the following non-limiting, exemplary genera: Anguina, Ditylenchus, Tylenchorizynchus, Pratylenchus, Radopholus, Hirschmannielia, Nacobbus, Hoplolaiinus, Scutellonema, Rotylenchus, Helicolylenchus, Rotylenchulus, Belonolaimus, Heterodera, other cyst nematodes, Meloidogyne, Griconemoides, Heinicycliophora, Paratylenchus, Tylenchulus, Aphelenchoides, Bursaphelenchus, Rhadinaphelenchus, Longidorus, Xiphinema, Tricliodorus, and Paratrichodorus, Dirofihiaria, Onchocerca, Brugia, Acanthocheilonema, Aelurostrongylus, Anchiostoma, Angiostrongylus, Ascaris, Bunostomum, C'apillaria, Chabertia, Cooperia, Crenosoina, Dictyocaulus, Dioctophyme, Dipetalonema, Dracunculus, Enterobius, Filaroides, Haeinonchus, Lagochilascaris, Loa, Manseonella, Muellerius, Necato; Nematodirus, Oesophagostomum, Ostertagia, Parafilaria, Parascaris, Physaloptera, Protostrongylus, Setaria, Spirocerca, Stephanogilaria, Strongyloides, Strongylus, Thelazia, Toxascaris, Toxocara, Trichinella, Trichostrongylus, Trichuris, Uncinaria, and Wuchereria.

Particularly preferred are nematodes including Dirofilaria, Onchocerca, Brugia, Acanthocheilonema, Dipetalonema, Loa, Mansonella, Parafilaria, Setaria, Stephanofilaria, and Wucheria, Pratylenchus, Heterodera, Meloidogyne, Paratylenchus. Species that are particularly preferred are: Ancylostoma caninum, I-Iaemonchus contortus, Trichinella spiralis, Trichurs muris, Dirofilaria immitis, Dirofilaria tenuis, Dirofilaria repens, Dirofilari ursi, Ascaris suum, Toxocara canis, Toxocara cati, Strongyloides ratti, Parastrongyloides trichosuri, Heterodera glycines, Giobodera pallida, Meloidogynejavanica, Meloidogyne incognita, and Meloidogyne arenaria, Radopholus siinilis, Longidorus elongatus, Meloidogyne hapla, and Pratylenchus penetrans.

In one embodiment, the invention is directed to a method for control of unwanted nematodes, the method comprising administering to vertebrates, plants, seeds or soil a pesticidal composition of this invention. In one embodiment, the nematodes are selected from the group consisting of: Anguina, Ditylenchus, Tvlenchorlzynchus, Pratylenchus, Radopholus, Hirschmanniella, Nacobbus, Hoplolaimus, Scutellonema, Rotylenchus, Helicotylenchus, Rotylenchulus, Belonolaimus, Heterodera, other cyst nematodes, Meloidog-vne, Criconemoides, Hemicycliophora, Paratylenchus, Tylenchulus, Aphelenchoides, Bursaphelenchus, Rhadinaphelenchus, Longidorus, Xiphinema, Trichodorus, and Paratrichodorus, Dirofihiaria, Onchocerca, Brugia, Acanthocheilonema, Aelurostrongylus, Anchiostoina, Angiostrongylus, Ascaris, Bunostomum, C'apillaria, C'habertia, Cooperia, renosoma, Dictyocaulus, Dioctophyme, Dip etaloneina, Dracunculus, Enterobius, Filaroides, Haemonchus, Lagochilascaris, Loa, Manseonelia, Muellerius, Necato,; Nematodirus, Oesophagostoinuin, Ostertagia, Parafuiaria, Parascaris, Physaloptera, Protostrongylus, Setaria, Spirocerca, Step hanogilaria, Strongvioides, Strongylus, Thelazia, Toxascaris, Toxocara, Trichinella, Trichostrongylus, Trichuris, Uncinaria, and Wuchereria. In a preferred embodiment, the nematodes are selected from the group consisting of: Dirofilaria, Onchocerca, Brugia, Acanthocheiloneina, Dipetalonema, Loa, Mansonella, Parafilaria, Setaria, Stephanofilaria, and JVucheria, Pratylenchus, Heterodera, Meloidogyne, Paratylenchus. In a preferred embodiment, the nematodes are selected from the group consisting of: Ancylostoina caninum, Haeinonchus contortus, Trichineila spiralis, Trichurs inuris, Dirofilaria iminitis, Dirofliaria tenuis, Dirojilaria repens, Dirofilari ursi, Ascaris suum, Toxocara canis, Toxocara cati, Strongyloides ratti, Parastrongyloides trichosuri, Heterodera glycines, Globodera pallida, Meloidogyne javanica, Meloidogyne incognita, and Meloidogyne arenaria, Radopholus similis, Longidorus elongatus, Meloidogyne hapla, and Pratylenchus penetrans. In most preferred embodiment, the nematodes are selected from the group consisting of: Heterodera glycines, Meloidogynejavanica, and Meloidogyne incognita.

Nematodes that attacks turf are also part of this invention. This includes, but not limited to, sting nematode (Belonolaimus longicaudatus), lance nematodes (Hoplolaimus species), stubby-root nematodes (Paratrichodorus species and related genera), spiral nematodes (Helicotylenchus, Scutellonema, and Peltamigratus species), ring nematodes (Criconemella species and related genera), root-knot nematodes (Meloidogyne species), awl nematode (Dolichodorus heterocephalus), St. Augustinegrass Cyst nematode (Heterodera leuceilyma). Other nematodes that may damage turf when especially numerous or when other pests, pathogens, or environmental conditions stress turf are also part of this invention. These include lesion (Pratylenchus), stunt (Tylenchorhynchus), dagger (Xiphinema), sheath (Hemicycliophora), and sheathoid (Hemicriconemoides) nematodes.

The composition described in this invention can be applied alone or in conjunction with substrate for a hydrogen-peroxide enzyme, chemical inducer of SAR and/or a substance that is encompassed by this invention. The composition may, for example, be applied simultaneously or sequentially.

The following examples further illustrate details for the method and compositions of this invention. The invention, which is set forth in the foregoing disclosure, is not to be limited either in spirit or scope by these examples. Those skilled in the art will readily understand that known variations of the conditions of the following procedures can be used.

Bioefficacy Assays

Boll Weevil Larvae Bioassay

Assays for activity against boll weevil larvae are carried out by incorporating the test sample into a agar liquid diet similar to that for southern corn rootworm [Marrone P. G., Ferri F. D., Mosley T. R., Meinke L. J., “Improvements in laboratory rearing of the southern corn rootworm, Diabrotica undecimpunctata howardi Barber (Coleoptera Chrysomelidae) on artificial diet and corn,” Journal of Economic Entomology, 78:290-3, 1985]. The test sample is substituted for the 20% water component. Neonate larvae are allowed to feed on the diet and mortality and growth stunting are evaluated.

Lepidopteran Larvae Bioassay

Lepidopteran larvae are tested on artificial diet treated with various amounts of oxalate for a number of days.

Boll Weevil Reproduction Test

Oxalate, in addition to lethal effects on larvae, will also affect the reproductive cycle of adult boll weevils that is demonstrated by the following study.

Preoviposition: Approximately 220 adult boll weevils, collected within 2 days of emergence, are divided into two groups. One is fed standard diet and the other is fed standard diet containing various concentrations of oxalate from Sigma. The adults are allowed to feed and mate for four days at which time mortality is determined.

Oviposition study: These two groups of adults are then divided into two subgroups and individually placed on artificial, oxalate-containing or control bolls. Artificial bolls are constructed of standard diet, with or without oxalate, and encased in paraffin containing 1% cottonseed oil. After three days at 27% C, the adults are removed and ten bolls from each of the four groups are removed and examined for eggs. The remaining bolls are incubated for an additional 7 days at 27% C to allow development of larvae. The bolls are then dissected and the eggs and larvae, dead and surviving, are counted.

Group 1=Control Adults placed on control bolls

Group 2=Control Adults placed on treated bolls

Group 3=oxalate-fed adults placed on control bolls

Group 4=oxalate-fed adults placed on treated bolls

Mode of Action Studies

The following studies are used to show that oxalate has a direct effect on the insect itself. Lepidopteran larvae and boll weevils are most susceptible to oxalate. Oxalic acid causes death in humans and animals due to its corrosive effects. In smaller amounts, oxalic acid causes a variety of pathological disorders, including hyperoxaluria, pyridoxine deficiency, cardiomyopathy, cardiac conductance disorders, calcium oxalate stones and renal failure.

Cotton Seed Diet Assay

Two treatment diets are made by mixing 30 g of one of two types of cottonseed flour into 170 mL of a 1.6% agar solution at 50% C, containing 0.13% propionic acid, 0.014% phosphoric acid, and 30 mg each of streptomycin sulfate and chlortetracycline. Before mixing, 10% KOH was used to adjust the pH to 6.2. One test diet utilized raw cottonseed flour (Sigma) as the nutrient source; the other utilized Pharmamedia.™. (Traders Protein), a flour made up of cottonseed embryos. The diets were incubated in a water bath at 40% C. Dilutions of the oxalate are incorporated into the diets as described above. Boll weevil larvae are allowed to feed and mortality rates are determined after six days. The results demonstrate that the enzyme is lethal to boll weevil larvae in the presence of cotton plant components.

Homogenized Cotton Leaf Tissue Assay

In order to test oxalate against boll weevil larvae in a host tissue diet environment, a study is conducted in which cotton leaf tissue is the only nutritional component of an agar-based diet. Two cotton leaves (each approx. 5 inches wide) with stems are homogenized at 50% C into 170 mL of a 1.6% agar solution containing 0.13% propionic acid, 0.014% phosphoric acid, and 30 mg each of streptomycin sulfate and chlortetracycline. Before addition of the leaves, 10% KOH is used to adjust the pH of the agar solution to 6.2. The leaf “diet” is allowed to cool to 40% C. Dilutions of oxalate and a water control are incorporated into the leaf “diet”, poured into insect diet trays and allowed to cool. Boll weevil eggs are added to the diet wells. The assay is evaluated six days later. The results will show that the oxalate maintains its insecticidal activity in the presence of cotton leaf tissue. This will illustrate that the oxalate is insecticidal in the presence of intact cotton tissue and cells.

Spectrum of Insecticidal Activity of Oxalate Three other coleopteran species, three other insects, and one mite species are evaluated for susceptibility to oxalate. Bioassays are evaluated after 4 to 7 days to measure acute effects of the oxalate on the insects' growth and survival. Mortality or stunting of larval growth are observed in these short term assays

The prior art documents mentioned herein are incorporated to the fullest extent permitted by law.

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting.

Reference is made to standard textbooks and other references (e.g., journal articles) that contain definitions and methods and means for carrying out basic techniques, encompassed by the present invention.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of killing insects, nematodes, fungi and/or other pathogens on plant tissue which comprises: applying a solution that includes a substance wherein the substance is a substrate for a hydrogen-peroxide generating enzyme, wherein the substrate is an oxalic acid, glucose, citrate or the like.

2. The method of claim 1 wherein the method further comprises applying a chemical inducer of systemic acquired resistance (SAR).

3. The method of claim 1 wherein the chemical inducer is hydrogen peroxide.

4. The method of claim 1 wherein hydrogen peroxide in the solution is present in a concentration of between 0.05 to 3.00% by weight.

5. The of claim 1 wherein the hydrogen peroxide in the solution has a concentration of between 0.05 and 1.5% by weight.

6. The of claim 1 wherein the method further includes at least one step of reapplying the solution.

7. The method of claim 1 wherein the solution is applied by hydraulic spraying.

8. The method of claim 1 wherein the solution is applied by misting.

9. The method of claim 1 wherein the solution is applied by fogging.

10. The method of claim 1 wherein the solution is applied by injecting the hydrogen peroxide into a commercial cooling system.

11. The method of in claim 1 wherein the solution is injected into a recirculating subirrigation nutrient system solution to control microbial growth in the irrigation water.

12. The method of claim 1 wherein the solution is applied to the plant tissue at a variety of stages in the plant production cycle.

13. The method of claim 1 wherein the solution is injected into the water that is misted over terminal stem cuttings until they reach root sufficiency.

14. The method of claim 1 wherein the step of applying said solution to plant tissue is repeated a plurality of times.

15. The method of claim 1, wherein the method further includes at least one step of reapplying the solution.

16. The method of claim 7 wherein the method further includes at least one step of reapplying the solution.

17. The method of claim 8 wherein the method further includes at least one step of reapplying the solution.

18. The method of claim 9 wherein the method further includes at least one step of reapplying the solution.

19. The method of claims 10 wherein the method further includes at least one step of reapplying solution.

20. The method of claim 11 wherein the method further includes at least one step of reapplying the solution.

21. The method of claim 12 wherein the method further includes at least one step of reapplying the solution.

22. The method of claim 13 wherein the method further includes at least one step of reapplying the solution.

23. The method of claim 1 wherein the method further comprises: applying a solution that includes a substance wherein the substance is a substrate for a hydrogen peroxide generating enzyme and the solution further includes an acid selected from the group consisting of acetic acid and phosphoric acid, wherein the substrate is an oxalic acid, glucose, citrate or the like.

24. The method of claim 1 wherein the method further comprises applying hydrogen peroxide to plant tissue, said hydrogen peroxide present in the solution at a concentration of between 0.05 and 1.5% by weight and the solution further includes an acid selected from the group consisting of acetic acid and phosphoric acid.

25. A method of controlling the larvae or grub of insects which comprise the southern masked chafer, the northern masked chafer, the Japanese beetle, the European chafer, the cupreous chafer, the oriental beetle and the May or June beetle which comprises contacting the larvae or grub in the soil with an insecticidally effective amount of a substance wherein the substance is a substrate for a hydrogen peroxide generating enzyme.

26. The method of claim 25 wherein the substrate for a hydrogen peroxide generating enzyme is selected from the group consisting of oxalic acid, glucose, citrate and the like.

27. The of claim 26 wherein the wherein the substrate is oxalic acid

28. The of claim 27 wherein the oxalic acid is applied in amount from about 0.001 to 100 kg per hectare.

29. The method of claim 28 wherein the oxalic acid is applied in an amount from about 0.01 to 10 kg per hectare.

30. The method of claim 1 wherein the insect is the Japanese beetle.

31. The method of claim 1 wherein the solution is applied to an area where turfgrass, ornamental plants or food crops are grown or will be grown.

32. The method of claim 31 wherein the solution is applied to turfgrass.

33. The method of claim 25 wherein the solution is applied as a constituent of a composition comprising an insecticidally effective amount of a compound and an agronomically acceptable carrier.

34. The method of claim 33 wherein the compound is present at from about 0.0001 to about 99% by weight of the composition.

35. The method of claim 32 wherein the agronomically acceptable carrier is a solid.

36. A pesticidal composition which comprises a pesticidally effective amount of a hydrogen peroxide generating substrate wherein the substrate is selected from the group consisting of oxalate, citrate, glucose, and the like, or a mixture thereof as the sole active ingredient, and a chemical inducer for systemic acquired resistance

37. The pesticidal composition of claim 36 wherein the chemical inducer is selected from the group consisting of hydrogen peroxide, SA, Jasmonic acid, INA, nitric oxide and mixtures thereof.
